(19) United States
(12) Patent Application Publication
(10) Pub. No.: US 2023/0035688 A1
(43) Pub. Date: Feb. 2, 2023

(54) METHOD FOR TREATING GASTRIC CANCER BY BLOCKING CCL28 CHEMOTACTIC PATHWAY

(71) Applicant: SHANGHAI JIAO TONG UNIVERSITY, Shanghai (CN)

(72) Inventors: Weiqiang GAO, Shanghai (CN); Bin MA, Shanghai (CN); Lu JI, Shanghai (CN)

(21) Appl. No.: 17/790,278

(22) PCT Filed: Sep. 4, 2020
(86) PCT No.: PCT/CN2020/113566

§ 371 Date: Jun. 30, 2022

(30) Foreign Application Priority Data
Dec. 31, 2019 (CN) 201911408316.3

(51) Int. Cl. C07K 16/24; G01N 33/574; G01N 33/68; A61P 35/00; A61P 1/00

(52) U.S. Cl. CPC C07K 16/24; G01N 33/57446; G01N 33/6872; A61P 35/00; A61P 1/00

(57) ABSTRACT

Provided is a method for treating gastric cancer by blocking a CCL28 chemotactic pathway. Specifically, provided is a use of a CCL28 gene or CCL28 protein for preparing a reagent or a kit for testing gastric cancer. The gastric cancer has an abnormal up-regulated expression of both a Wnt/β-catenin signaling pathway and molecules related to CCL28 chemokine. Also provided are a testing kit, a use of a pharmaceutical composition, and a method for inhibiting the growth of cancer cells in vivo and in vitro.

### TECHNICAL FIELD

The invention belongs to the field of biomedicine, in particular, the invention relates to a method for treating gastric cancer by blocking CCL28 chemotactic pathway

### BACKGROUND

The rapid developing immunotherapy has brought hope to countless tumor patients. The molecular characteristics of gastric cancer indicate that gastric cancer with EBV-related subtypes shows increased PD-L1 expression, suggesting that anti-PD immunotherapy may be effective in these patients. However, compared with melanoma and lung cancer, due to the complexity of the immune microenvironment and molecular mechanism of gastric cancer, the development of these immunotherapy methods in gastric cancer encountered many practical problems. The immune escape mechanism of tumor is also a major obstacle to tumor immunotherapy. Regulatory T cells (Treg) are a kind of important immunosuppressive cells involved in tumor immune escape. Increase of these cells will lead to a decrease in the function and number of anti-tumor effector immune cells, and also lead to a decrease or even loss of the efficacy of immunotherapy or other therapies of tumor. For patients suffering from gastric cancer and other immunotherapy for a long time, low immunity and derived diseases thereof are important causes of death.

Therefore, there is an urgent need in the art for an effective treatment regimen of refractory gastric cancer while reducing side effects.

### SUMMARY OF THE INVENTION

The purpose of the present invention is to provide an effective treatment regimen of refractory gastric cancer while reducing side effects.

In the first aspect of the invention, provided is a use of a CCL28 gene or CCL28 protein or a detection reagent thereof for preparing a reagent or a kit for detecting gastric cancer, the gastric cancer is a type of gastric cancer wherein expressions of Wnt/β-catenin signal pathway and chemokine CCL28 related molecule are abnormal up-regulated at the same time.

In another preferred embodiment, the pathological manifestations of the gastric cancer have one or more pathological manifestations selected from the group consisting of:

(1) up-regulated Wnt/β-catenin signaling pathway-related expression in gastric tissues;

(2) up-regulated chemokine CCL28 related expression in gastric tissue;

(3) gastric tumor;

(4) loss of parietal cells; and

(5) increase of regulatory T cells (Treg) of gastric tissues.

In another preferred embodiment, the pathological manifestations of the gastric cancer further include low immune response of the body.

In another preferred embodiment, the gastric cancer comprises an in situ gastric cancer, an intestinal gastric cancer, and a diffuse gastric cancer.

In another preferred embodiment, the gastric cancer comprises Helicobacter pylori-negative gastric cancer.

In another preferred embodiment, the gastric tissue comprises gastric cancer tumor tissue, gastric para-carcinoma tissue, or a combination thereof.

In another preferred embodiment, the Wnt/β-catenin signaling pathway related expression is selected from the group consisting of the expression level of the transcription factor TCF1, the expression level of the transcription factor TCF4, the content of β-catenin, the level of β-catenin nuclear ectopia, the transcriptional activity of β-catenin/TCF4 complex, the level of GSK3β phosphorylation, or combinations thereof.

In another preferred embodiment, the chemokine CCL28 related molecule comprises CCL28 gene (including the nucleotide sequence, cDNA sequence, and/or mRNA of the genome), the gene of CCL28 receptor includes the nucleotide sequence, cDNA sequence, and/or mRNA of the genome, CCL28 proteins, CCL28 receptor proteins, or combinations thereof.

In another preferred embodiment, the CCL28 receptor comprises CCR3, and/or CCR10.

In another preferred embodiment, the kit includes: reagents for quantitative detection of the protein or mRNA of the CCL28-related molecule and the corresponding label or instruction.

In another preferred embodiment, the reagent comprises specific primer, specific antibody, probe and/or chip of the CCL28-related molecule.

In another preferred embodiment, the reagent includes: a chip for detection, such as a nucleic acid chip and a protein chip.

In another preferred embodiment, the nucleic acid chip comprises a substrate and specific oligonucleotide probe for cancer-related gene sampled on the substrate, and the specific oligonucleotide probe for cancer-related gene comprises a probe that specifically binds to a CCL28-related gene or mRNA.

In another preferred embodiment, the protein chip comprises a substrate and a specific antibody for cancer-related protein sampled on the substrate, and the specific antibody for cancer-related protein comprises a specific antibody of anti-CCL28-related protein.

In another preferred embodiment, the CCL28-related protein comprises a fusion protein and a non-fusion protein.

In another preferred embodiment, the reagent or the kit comprises CCL28 gene (or nucleic acid molecule) or CCL28 protein as a standard.

In another preferred embodiment, the CCL28 gene (or nucleic acid molecule) or CCL28 protein comprises a wild type and/or a mutant type.

In another preferred embodiment, the reagent or the kit further comprises a detection reagent for detecting CCL28 gene or CCL28 protein.

According to a second aspect of the present invention, provided is a kit for detecting gastric cancer, the kit comprises a container containing a detection reagent for detecting CCL28-related protein or mRNA; and a label or instruction indicating that the kit is used for detecting gastric cancer.

In another preferred embodiment, the gastric cancer is a type of gastric cancer wherein both Wnt/β-catenin signal pathway and chemokine CCL28 related molecule expression are abnormal up-regulated at the same time.

In another preferred embodiment, the contents indicated in the label or the instruction are selected from the group consisting of:

a) when the ratio of the mRNA expression A1 of the CCL28-related protein of a testing subject to the mRNA expression A0 of the CCL28-related protein of the para-carcinoma tissue (A1/A0) is ≥2, indicating that the probability of gastric cancer of the testing subject is higher than that of the general population;

b) when the ratio of the mRNA expression of the CCL28-related protein of a testing subject to the mRNA expression of the CCL28-related protein of the para-carcinoma tissue A1/A0 is ≥2, the higher A1/A0 ratio indicating the higher malignant degrees of the gastric cancer of the testing subject;

c) when the ratio of the mRNA expression of the CCL28-related protein of a testing subject to the mRNA expression of the CCL28-related protein of the para-carcinoma tissue A1/A0 is ≥2, the higher A1/A0 ratio indicating poorer prognosis and higher metastasis rate of the gastric cancer of the testing subject;

In another preferred embodiment, the detection reagent comprises specific primer, specific antibody, probe and/or chip.

In another preferred embodiment, the kit is used for detecting an in vitro human tumor tissue sample or blood sample.

In another preferred embodiment, the tumor tissue sample is a gastric cancer sample.

According to a third aspect of the present invention, provided is a use of a CCL28 inhibitor for preparing a pharmaceutical composition for inhibiting the growth or proliferation of cancer cells, or for preparing a pharmaceutical composition for treating gastric cancer; the gastric cancer is a type of gastric cancer wherein both Wnt/β-catenin signal pathway and chemokine CCL28 related molecule expression are abnormal up-regulated at the same time.

In another preferred embodiment, the inhibitor is selected from the group consisting of: an antibody or small molecule inhibitor targeting CCL28 and/or the receptor protein thereof; a targeting nucleic acid molecule or gene editor targeting CCL28 and/or receptor gene thereof; or combinations thereof.

In another preferred embodiment, the CCL28 receptor comprises CCR3, and/or CCR10.

In another preferred embodiment, the antibody is selected from the group consisting of a polyclonal antibody, a monoclonal antibody, a chimeric antibody, a bispecific antibody, an antibody conjugate, a small molecule antibody, an antibody fusion protein, and combinations thereof.

In another preferred embodiment, the small molecule antibody is selected from the group consisting of a single chain antibody ScFv, a Fab antibody, an Fv fragment, and combinations thereof.

In another preferred embodiment, the ScFv antibody comprises a secretory single chain antibody expressed (including overexpression) in therapeutic cells.

In another preferred embodiment, the therapeutic cells comprise mesenchymal stem cells, CAR-T cells.

In another preferred embodiment, the vector comprises a bacterial plasmid, a phage, a yeast plasmid, a plant cell virus, a mammalian cell virus such as an adenovirus, a retrovirus, or other vectors.

In another preferred embodiment, the inhibitor is selected from the group consisting of a plant extract inhibitor, a small molecule compound inhibitor, a nucleic acids inhibitor, a peptides inhibitor, a polysaccharides inhibitor, a viral vector inhibitor, a liposome vector inhibitor, or a nanoparticle vector inhibitor.

In another preferred embodiment, the pharmaceutical composition further comprises other Wnt/β-catenin signaling pathway inhibitors.

In another preferred embodiment, the pharmaceutical composition is capable of synergistically inhibiting abnormal activation of Wnt/β-catenin signaling pathway in tumor tissue.

In another preferred embodiment, the pharmaceutical composition is capable of inhibiting the infiltration of regulatory T (Treg) cells into tumor tissue while increasing the activity of peripheral immunity.

In a fourth aspect of the present invention, provided is an in vitro non-therapeutic method for inhibiting the growth or proliferation of cancer cells, comprising the step of: culturing cancer cells in the presence of a CCL28 inhibitor, thereby inhibiting the growth or proliferation of the cancer cells.

In another preferred embodiment, the method comprises adding a CCL28 related molecule inhibitor to a cancer cell culture system, thereby inhibiting the growth or proliferation of the cancer cells.

In another preferred embodiment, the cancer cells are gastric cancer cells.

In another preferred embodiment, the gastric cancer cell is selected from the group consisting of AGS cell lines, SC7901 cell lines, AZ-521 cell lines, primary gastric cancer cells, or combinations thereof.

In a fifth aspect of the present invention, provided is a method for screening candidate compounds for treating cancers, including the steps of:

(a) in a testing group, adding a testing compound to the cell culture system, and observing the expression and/or activity of CCL28-related molecules in the cells of the testing group; in a control group, not adding the testing compound to the culture system of the same cell, and observing the expression and/or activity of CCL28-related molecules in the cells of the control group;

wherein, if the expression and/or activity of CCL28-related molecules of the cells of the testing group is lower than that of the control group, indicating that the testing compound is a candidate compound for treating cancer having inhibitory effect on the expression and/or activity of CCL28-related molecules.

In another preferred embodiment, the cells comprises: cancer cells or normal cells;

In another preferred embodiment, the cells are gastric cancer cells or gastric cells.

In another preferred embodiment, the method further comprises the following steps:

(b) for the candidate compound obtained in step (a), further testing its inhibitory effect on the growth or proliferation of cancer cells.

In another preferred embodiment, the step (b) comprises the steps of: in the testing group, adding a testing compound to the culture system of cancer cells, and observing the number and/or growth state of cancer cells; in the control group, not adding the testing compound to the culture system of cancer cells, and observing the number and/or growth state of cancer cells; wherein, if the number and/or growth rate of cancer cells of the testing group is less than that of the control group, indicating that the testing compound is a candidate compound for treating cancer having inhibitory effect on the growth or proliferation of cancer cells.

In the sixth aspect of the present invention, provided is a method for inhibiting or treating gastric cancer, comprising the steps of: administering a safe and effective amount of the CCL28 related molecule inhibitor to a subject (mammalian) in need; the gastric cancer is a type of gastric cancer wherein both Wnt/β-catenin signal pathway and chemokine CCL28 related molecule expression are abnormal up-regulated at the same time.

It should be understood that within the scope of the present invention, the above technical features of the present invention and the technical features specifically described in the following (e.g., embodiments) can be combined with each other, thereby forming a new or preferred technical solution. Due to space limitations, it will not be repeated herein.

### DESCRIPTION OF THE DRAWINGS

FIG. 1 shows that the β-catenin signaling pathway induces CCL28 expression in gastric cancer. Figure A shows the qPCR detection of chemokine in a gastric cancer cell line SGC7901 overexpressing β-catenin. In the figure, B shows the detection of β-catenin and CCL28 protein levels in gastric cancer cell lines SGC7901 and AGS which overexpress or knock down β-catenin. In the figure, C and D are the immunohistochemical detection of β-catenin and CCL28 in the tissue chip of gastric cancer patients, and results of the positive rate correlation analysis.

FIG. 2 shows that CCL28 is a target gene for direct transcriptional regulation of the β-catenin signaling pathway. In the figure, A is a schematic diagram of the binding site of the β-catenin/TCF transcription factor complex in the CCL28 gene promoter. In the figure, B shows that chromatin immunoprecipitation experiments have proved that β-catenin binds to the predicted sites 1 and 3 in the CCL28 gene promoter. In the figure, C shows that overexpression of β-catenin upregulates the activity of CCL28 promoter, and the mutation of core sequence of binding sites 1 and 3 (CCL28.mut) lead to the lose of the regulatory effect of β-catenin. In the figure, D shows that overexpression of β-catenin upregulates the activity of Wnt pathway reporter gene TOPflash and CCL28 promoter. Treatment with Wnt inhibitor iCRT14 has reduced the activity of both reporter genes. In the figure, E shows that, using RNA interference, compared with control shRNA sequence shScr, after their expression being knocked down by shRNA targeting Wnt pathway transcription factors TCF/LEF family members TCF1(shTCF1) and TCF4(shTCF4), the activity of CCL28 promoter decreases, while knocking down LEF1 expression (shLEF1) do no effect on the activity of CCL28 promoter. Figure F shows that overexpression of transcription factors TCF1 and TCF4 up-regulate the activity of CCL28 promoter, while overexpression of LEF1 has no effect on the activity of CCL28 promoter. Which shows that transcription factors TCF and TCF4 synergize with β-catenin to regulate the expression of CCL28.

FIG. 3 shows that the Wnt pathway inhibitor iCRT14 reduces CCL28 expression in mouse gastric cancer. In the mouse orthotopic gastric cancer model induced by H. felis/MNU, it is detected by immunoblotting (A) and ELISA (B) that the expression of CCL28 protein in the stomach of mice treated with Wnt pathway inhibitor iCRT14 is significantly reduced. In the figure, Vehicle represents the drug solvent (medium).

FIG. 4 shows that the expression of CCL28 is associated with the development of gastric cancer. In the figure, A shows that Oncomine database analysis shows the expressions of CCL28 mRNA in Intestinal Type and Diffuse Type gastric cancer are higher than that in normal tissues. In the figure, B shows the immunohistochemical analysis of tissues from gastric cancer patients has shown that the expression level of protein of CCL28 is higher in grade II and III of gastric cancer which having higher deterioration than in grade I. *P<0.05; ***P<0.001.

FIG. 5 shows that gastric cancer cells activated by the β-catenin signaling pathway induce the expression of CCL28 to achieve the migration of regulatory T cells. In the figure, A is western blot analysis of β-catenin and CCL28 in SGC7901 cell line with simultaneous overexpression of β-catenin (Vector) and knockdown of CCL28 (shScr). In the figure, B is a flow cytometry pattern of regulatory T cells (Tregs), CD4⁺ T cells, and CD8⁺ T cells. In the figure, C and D are the statistical results of the proportion and absolute number of regulatory T cells (Tregs), CD4⁺ T cells and CD8⁺ T cells. *P<0.05; **P<0.01.

FIG. 6 shows that CCL28 antibody treatment inhibits the progression of H. felis/MNU-induced in situ gastric cancer in mice. In the figure, A is a diagram of CCL28 antibody treatment. In the figure, B shows gastric anatomy and tumor size statistics for CCL28 antibody treatment. *P<0.05. In the figure, C shows that HE staining, Alcian blue staining and H+K+ATPase immunohistochemical staining of CCL28 antibody treatment. In the figure, D is the pathological statistical results of the sites of corpus gastricum (Corpus) and gastric antrum (Antrum). *P<0.05; **P<0.01; ***P<0.001.

FIG. 7 shows that CCL28 antibody treatment alleviates the inhibition of the immune microenvironment. In the figure, A is a flow cytometry analysis chart and statistical chart of regulatory T cells (Tregs). In the figure, B shows flow cytometry analysis chart and statistical chart of IFNγ⁺CD4⁺ T cells. In the figure, C shows flow cytometry analysis chart and statistical chart of IFNγ⁺CD8⁺ T cell. *P<0.05; ***P<0.001.

FIG. 8 shows that blocking with CCL28 antibody has no significant therapeutic effect in melanoma and breast cancer xenograft tumor models. In the figure, A shows the growth curve of melanoma B16 subcutaneous xenograft tumor. In the figure, B shows the growth curve of breast cancer 4T1 subcutaneous xenograft tumor. In the figure, C shows the survival rate of tumor-bearing mice with melanoma B16. In the figure, D shows the survival rate of tumor-bearing mice with breast cancer 4T1.

FIG. 9 shows the expression level of CCL28 protein in human tissues. It is found in The Human Protein Atlas database that CCL28 protein highly expresses in the brain, gastrointestinal tract, and pancreas.

### DETAILED DESCRIPTION OF THE INVENTION

After extensive and in-depth research, the present inventors have unexpectedly discovered for the first time that Wnt/β-catenin up-regulates the expression of CCL28 in gastric cancer cells and then increases the regulatory T cells in tumors. Blocking the CCL28 chemotactic pathway can effectively reduce the infiltration of regulatory T cells in tumors and inhibit tumor development. Further research has found that such therapeutic effects is only on gastric cancer models, and there is no obvious effect on other solid tumors such as melanoma and breast cancer.

The inventor has completed the present invention on this basis.

### Description of Terms

Unless otherwise defined, all technical and scientific terms used herein have the same meaning as would normally be understood by those of ordinary skill in the art to which the invention belongs.

As used herein, term “about” means that the value may change by no more than 1% from the enumerated value when used in reference to a specific enumerated value. For example, as used herein, the expression “about 100” includes all values between 99 and 101 and (e.g., 99.1, 99.2, 99.3, 99.4, etc.).

As used herein, term “contain” or “include (comprise)” may be open, semi-closed, and closed. In other words, said term also includes “consisting essentially of” or “consisting of”.

Chemokines and CCL28

Chemokines are a kind of cytokines that regulate cell migration which play a vital role in the formation of tumor microimmune environment and the occurrence and development of tumors.

C— C motif chemokine ligand 28 (C—C motif chemokine ligand 28, CCL28), located at 5p12, encoding 8 exons. CCL28 is a chemokine that mediates the migration of cells expressing its receptor CCR3 or CCR10.CCL28 is expressed in epithelial cells of digestive tract, lung, breast and other tissues (as shown in FIG. 9).

The present invention demonstrates for the first time that the Wnt/β-catenin-CCL28 pathway is up-regulated in the pathological process of gastric cancer, and also confirms for the first time the therapeutic effect of blocking with CCL28 antibody in the gastric cancer model.

In the present invention, the abnormal up-regulation of the expression of chemokine CCL28-related molecule refers to the ratio of the mRNA expression C1 of CCL28-related molecules in a certain tissue (such as gastric cancer tissue) to the mRNA expression C0 of CCL28-related molecules in normal tissues (such as para-carcinoma) (C1/C0)≥1.2, preferably ≥1.5, more preferably ≥2.0 or ≥2.5.

Wnt/β-Catenin Pathway

In cells, the level of β-catenin in cytoplasm is strictly controlled by a polyprotein disruption complex composed of APC, Axin and glycogen synthase kinase-3β (GSK-3β). The polyprotein disruption complex can phosphorylate β-catenin, and further induce ubiquitination and proteasome thereof—mediated degradation. The stimulation of Wnts pathway inhibits the activity of the disruption complex, breaks the stability of cytosolic β-catenin, and cytosolic β-catenin translocates to the nucleus and activates transcription together with the LEF/TCF family. This activation program of β-catenin transcription target is the catenin response transcription (CRT) effect, which is a key aspect of stimulus responsiveness of cells to specific Wnt.

Most, if not all, of Wnt-related carcinogenesis is caused by CRT misregulation, thus targeting β-catenin-TCF/LEF complex becomes an ideal therapeutic target. Wnt/β-catenin pathway is one of the most common oncogene pathways in intestinal cancer, gastric cancer and other types of cancer. It plays an important role in the occurrence and development of cancer, and is also one of the most interesting targets in cancer therapy. In the classical Wnt signaling pathway, CTNNB1 and APC are common genes significantly mutated. Taking gastric cancer as an example, about 70% of gastric cancer patients are accompanied with mutations in Wnt/β-catenin signaling pathway. In addition to gene mutations, changes in components of many Wnt signaling pathway may be achieved by up-regulating positive regulators or down-regulating negative regulators, and ultimately lead to activation of the classical Wnt pathway. Studies showed that Helicobacter pylori infection can result in the activation of Wnt/β-catenin signaling pathway in gastric epithelial cells. Summing up, these findings suggest a critical role of the Wnt/β-catenin signaling pathway in the pathogenesis of cancer.

Abnormal Wnt/β-catenin signaling pathway is found in most cancer types and is a very important oncogene pathway in occurrence and development of tumors. Through the analysis of a variety of tumor samples, the increased expression of β-catenin has a negative correlation with the infiltration of lymphocytes in the tumor, suggesting that Wnt/β-catenin also leads to immune evasion and tolerance to immunotherapy of tumors.

The invention proves for the first time that in the pathological process of gastric cancer, β-catenin and the transcription factor TCF/LEF complex can up-regulate the expression of CCL28 in gastric cancer by binding to the CCL28 promoter region, and such up-regulated expression of Wnt/β-catenin-CCL28 pathway in the pathological process of gastric cancer is unique. In one embodiment of the present invention, using the same treatment manner and dosage, the CCL28 antibody significantly relieves the progression of gastric cancer, while has no similar effect on other solid tumors such as melanoma and breast adenoma.

In the present invention, the abnormal up-regulation of Wnt/β-catenin signal pathway related molecule expression refers to the ratio of the mRNA expression B1 of Wnt/β-catenin signal pathway related molecule in a certain tissue (such as gastric cancer tissue) to the mRNA expression B0 of Wnt/β-catenin signal pathway related molecule in normal tissues (such as para-carcinoma) (B1/B0)≥1.2, preferably ≥1.5, more preferably ≥2.0 or ≥2.5.

Wnt/β-Catenin Signaling Pathway Inhibitor iCRT14

The Wnt/β-catenin signaling pathway inhibitor used herein is iCRT14.iCRT14 is a catenin responsive transcription inhibitor.

In the initial study (An RNAi-based chemical genetic screen identifies three small-molecule inhibitors of the Wnt/wingless signaling pathway. Gonsalves et al. Proc. Natl. Acad. Sci. USA, 2011; 108:595), 34 molecules having statistically significant inhibitory effect on dTF12-luciferase reporter gene activity were identified by preliminary screening of the experiment (the total hit rate is about 0.3%), these compounds are called CRT inhibitors (iCRT).

iCRT14 is a β-catenin responsive transcription inhibitor belonging to thiazolidinediones and is a Wnt/β-catenin pathway inhibitor. Empirical molecular formula (Hill notation) C₂₁H₁₇N₃O₂S. It is a potent inhibitor of the catenin β-catenin and the transcription factor TCF/LEF complex. It can disrupt the β-catenin-TCF/LEF interaction in a dose-dependent manner and cause G0/G1 arrest in colon tumor cell lines, leading to a sustained weakening of cell proliferation and tumor growth reduction in colon cancer cells.

It is found for the first time by the present invention that in gastric cancer, the regulatory factors TCF1 and TCF4, not LEF1, synergized with β-catenin regulates the transcription of CCL28 gene.

S33Y.β-Catenin

In the present invention, the purpose of overexpressing β-catenin is achieved by transferring S33Y.β-catenin plasmid into gastric cancer cells to inhibit β-catenin phosphorylation.

The DNA plasmid of S33Y.β-catenin refers to a DNA plasmid carrying a fragment that serine (S33) at position 33 of the β-catenin amino acid sequence is mutated to tyrosine (Y). S33 is the phosphorylation site of GSK30. After this site being phosphorylated, β-catenin enters the degradation pathway. Mutant S33Y.β-catenin after S33 being mutated to tyrosine (Y) can escape phosphorylation and degradation pathways, and then enrich more efficiently in cells. This mutation was also detected in samples from some patients with gastrointestinal tumors. Which is used in the invention for studying the role of abnormal activation of Wnt/β-catenin pathway in tumor cells.

H. felis/MNU-Induced Gastric Cancer Model

Helicobacter pylori is considered to be one of the main causes of chronic gastritis. Therefore, the establishment of gastric cancer mouse model using Helicobacter pylori is of great significance in studying the occurrence and development of human gastric cancer. C57BL/6 mice have significant resistance to the colonization of various Helicobacter pylori strains in the stomach. Therefore, it is very important to use H. felis (a close relative of Helicobacter pylori) to establish a mouse model of gastric cancer. This strain is isolated from the stomach of cats and it is easy to be colonized in the stomach of mice. Which can induce severe gastritis and atrophy in mice. Manifestations of felis-infected mice are gastric SPEM, dysplasia and invasive tumor, and longer observation period. In this model, extensive dysplastic lesions can be observed at the squamous columnar junction (SCJ) of the gastric corpus along gastric lesser curvature, with the development of large polypoid gastric antral tumors.

By means of study of the mouse model infected with helicobacter, the researchers determined the influence of other cofactors in the occurrence of gastric cancer, such as sex, diet and concomitant infection. The use of N-methyl-N-nitrosourea (MNU) before Helicobacter pylori infection can induce more severe precancerous lesions and increase the incidence of gastric cancer. MNU plays an important role in inducing gastric carcinogenesis in mouse models. Twice-a-week intragastric administration (0.5 mg MNU) can lead to the death of most Balb/c mice due to squamous cell carcinoma of the anterior stomach. Before the use of MNU, surgical resection of the anterior stomach is helpful to promote the occurrence of well differentiated adenocarcinoma of the glandular stomach, and the incidence rate is 100% after 40 weeks of use. Therefore, the gastric gland body is very sensitive to the carcinogenic effect of MNU. 240 ppm MNU was dissolved in drinking water and administered continuously for 5 weeks (every other week). The results of experiments in 6 strains of mice shows that this method can induce gastric cancer. Therefore, this project uses the method of combination of H. felis infection and MNU administration to establish a mouse gastric cancer model.

Immune Evasion

Immune evasion has been recognized as a new symbol of cancer. Understanding of tumor immune microenvironment is essential for discovering new therapeutic targets and predicting immunotherapy responses. A normal immune system will suppress the occurrence and development of tumors. With the progress of cancer, tumors will produce many immune evasion mechanisms, leading to the strengthening of tumor growth.

Data from mouse models and tumor patient samples show that myeloid suppressor cells (MDSCs) and M2 macrophages are involved in immunosuppression of gastric cancer. Regulatory T (Treg) cells are a class of immunosuppressive cells that are found to be involved in accelerating tumor progression in a variety of cancer types. Regulatory T cells (Treg) are a kind of important immunosuppressive cells involved in tumor immune evasion. Increase of these cells will lead to a decrease in the function and number of anti-tumor effector immune cells, and also lead to a decrease or even loss of the efficacy of immunotherapy or other therapies of tumor.

In a preferred embodiment of the present invention, the use of CCL28 antibody can effectively inhibit the migration process of targeting regulatory T cells to tumors without affecting the apoptosis of regulatory T cells. At the same time, only targeting Treg cells in gastric cancer tumor tissue and spleen without reducing the ratio of Treg cells in blood can also improve the overall immune response.

The Technical Solution of the Present Invention has the Following Advantages:

1. The present invention provides a method of treating gastric cancer with simultaneous abnormally elevated Wnt/β-catenin signaling pathway-CCL28 by inhibiting CCL28. The use of CCL28 antibody treatment provided by the present invention can significantly reduce the tumor size, significantly change in developmental abnormalities and intestinal transformation, and significantly alleviate the phenomenon of normal cell loss.

2. The present invention has found that the β-catenin/CCL28 has a positive correlation with occurrence and deterioration of gastric cancer, thus providing a detection method for judgment of diagnosis, screening and prognosis of gastric cancer, which can screen gastric cancer incidence in the population and accurately detect metastasis of gastric cancer.

3. The present invention inhibits β-catenin/CCL28, whose effects only targeting Treg cells in gastric cancer tumor tissue and spleen, without reducing the ratio of Treg cells in blood, and can improve the overall immune response.

4. The method of treating malignant gastric cancer by blocking the CCL28 chemotactic pathway according to the present invention targets the migration process of regulatory T cells to the tumor and does not affect the apoptosis of the regulatory T cells, which may provide a more novel and effective means for the immunotherapy that targets regulatory T cells.

5. The present invention provides therapeutic thoughts of inhibiting Wnt/β-catenin signaling pathway in tumor cells by inhibiting CCL28.

6. It is found in the present invention that gastric cancer with simultaneous abnormally elevated Wnt/β-catenin signaling pathway-CCL28 is a disease phenotype that develops significantly different from other tumors, even from solid tumors. The main manifestation is that blocking CCL28 chemotactic pathway can effectively inhibit the development of malignant gastric cancer, but the effect on other tumor models is not clear, especially there is no effect on melanoma cell B16 and breast cancer cell 4T1 models.

The present invention was further described hereafter in combination with specific examples. It should be understood that these examples are only used to illustrate the and not to limit the scope of the invention. The experimental methods with no specific conditions described in the following examples are generally performed under the conventional conditions, for example, according to J. Sambrook et al., Molecular Cloning: A Laboratory Manual, 3rd Edition, Science Press, 1989, or according to the manufacture's instructions. Unless otherwise stated, percentages and parts are by weight.

General Experimental Method

1. Cell Culture

Human gastric cancer cell lines AGS and SC7901 were cultured in RPMI 1640 (Life Technologies) medium containing 10% serum (Gbico) and 1% penicillin/streptomycin double antibody (Life Technologies).

2. RNA Purification and Quantitative PCR

Total RNA from cell lines and mouse gastric tissues were extracted using RNeasy Mini Kit (Qiagen), followed by reverse transcription synthesis of cDNA using PrimeScript RT Reagent Kit (Takara). QPCR was performed using SYBR Green PCR Master Mix kit (Takara) and ABI 7900HT Fast Real-Time PCR System (Applied Biosystems). The quantitative calculation uses the method of ΔΔCt.

3. Western Blotting

The proteins in gastric cancer cell line and mouse gastric tissue were lysed with RIPA lysate (Thermo Scientific) added with protease inhibitor (Roche), and the protein concentration was detected using BCA Protein Assay Reagent (Thermo Scientific). The detected proteins were as follows: β-catenin (Abcam), CCL28 (R & D Systems), active β-catenin (Merck), GAPDH (Abcam) and β-tubulin (Abcam).

4. Detection of Luciferase Reporter Gene Activity

Wnt/β-catenin reporter plasmid M50 Super 8X TOPFlash (Addgene plasmid #12456) and mutant control M51 Super 8X FOPFlash (Addgene plasmid #12457) are gifts from Randall Moon. The promoter of the human CCL28 gene (2.8 kb, −2576/+205 relative to the transcriptional start site) was cloned into the firefly luciferase reporter gene construct pGL4. Mutations of potential TCF/LEF binding sites on the CCL28 promoter were introduced using the Hieff Mut™ Multisite Directed Mutagenesis Kit (Yeasen, Shanghai) to produce a mutant CCL28 promoter report construct (pGL4-CCL28.mut). The plasmids were transfected using a jetPRIME transfection reagent (Polybus Transfection). Transfection efficiency was standardized by co-transfection with a pRL-CMV reporter gene containing the Ranilla Luciferase gene. Firefly and Ranilla luciferase activities were measured using a Dual-Glo luciferase assay system (Promega).

5. Chromatin Immunoprecipitation ChIP

SGC7901 cells were fixed with 1% formaldehyde solution for 15 minutes, and then the reaction was stopped by adding 0.125M glycine solution. The reaction solution was washed off with PBS, and then Farnham lysate was added to collect cells. The cell mass was centrifugally collected, the cells was resuspended into RIPA lysate, and the ultrasound instrument was used to break DNA in the cells. The DNA fragments were incubated overnight with a previously coupled antibody and Dynabeads (purchased from ThermoFisher) complex at 4 degrees under rotation. The antibodies were anti-β-catenin and control IgG (Abcam). The DNA-protein-antibody-Dynabeads complex was precipitated by magnet, and finally the DNA fragments were eluted by buffer. Primers were designed for quantitative PCR at three different β-catenin potential binding sites on the CCL28 promoter to check the content of DNA fragments at three sites in the sample. The relative enrichment was calculated as the relative binding of β-catenin to control IgG at the potential DNA binding site.

6. Experiment of Isolation and Migration of Human Peripheral Blood Mononuclear Cells

Fresh peripheral blood was separated by gradient centrifugation using Ficoll-Paque Plus (purchased from GE Healthcare), then resuspended into individual cells with PBS and counted using a blood cell counting dish.

Supernatant of culture medium of SGC7901 gastric cancer cell line was plated on the lower layer of 8-μm-pore transwell chambers (purchased from Corning), and 2 million peripheral blood mononuclear cells resuspended in PBS containing 1% serum were plated in the small chamber. Placed in an incubator at 37° C. for 6 hours, PBMC migrating to the lower layer was collected and regulatory T cells, CD4⁺ and CD8⁺ T cells were detected by flow cytometry. The antibodies used were as follows: CD3 [SK7] (purchased from eBioscience), CD4 [RPA-T4] (purchased from eBioscience), CD8a [OKT8] (purchased from eBioscience), CD25 [BC96] (purchased from BioLegend) and FOXP3 [206D](purchased from BioLegend).

7. In Situ Gastric Cancer Mouse Model and Therapeutic Regimen

In order to establish gastric cancer mouse mode induced by Helicobacter felis (H. felis) and N-Methyl-N-nitrosourea (MNU), 8-week-old wild-type mice were fed by gavage with H. felis strain (ATCC 49179) three times a week, followed by given drinking water containing 240 ppm MNU every other week for a total of 12 weeks.

Antibody therapeutic regimen: intraperitoneal injection of 50 mg/kg CCL28 monoclonal antibody (purchased from R & D Systems), the control group used the isotype control IgG antibody of this antibody, please refer to FIG. 4A for the specific time.

8. Flow Cytometry Analysis

In order to detect immune cells in peripheral blood, spleen and gastric tissues, a tissue homogenizer gentle MACS Octo dissociator (purchased from Miltenyi Biotec) was used for cell isolation of spleen and stomach. Gastric tissues were decomposed with 1 mg/ml collagenase IV (purchased from Thermo Fisher) and 50 μg/ml (20 U/ml) DNase I (stock: 5 mg/ml) (purchased from Sigma-Aldrich), and erythrocytes in the tissues were removed using Red Blood Cell Lysis Buffer (purchased from eBioscience). Antibodies used in flow cytometry detection were purchased from BioLegend or eBioscience companies. For intracellular antigen detection, cell activator (purchased from BioLegend) should be used for 6 hours of stimulation. Cyto-Fast™Fix/Perm Buffer Set (purchased from BioLegend) was used for Intercellular antigen staining. Nuclear proteins were treated using Foxp3/Transcription Factor Staining Buffer Set (purchased from BD Biosciences). Flow cytometry data was processed by FACS Aria II cytometer (purchased from BD Biosciences) and FlowJo software.

9. Immunohistochemistry

Tissue paraffin sections of gastric cancer patients were purchased from Alenabio Company. Mouse gastric tissues were embedded in paraffin wax and cut into 5 micron tissue sections, dewaxed and performed antigen retrieval. After blocking, using the following antibodies of β-catenin (purchased from Abcam), H+K+ATPaseβ (ATP4B) (purchased from Abcam), FOXP3 (purchased from Abcam), CCL28 (purchased from R&D Systems) for primary antibodies at 4 degrees overnight, incubated with secondary antibody on the second day, and DAB Peroxidase Substrate Kit (purchased from Gene Tech, Shanghai) was used to develop color. Finally, hematoxylin was used to dye the nucleus, and rehydrated and sealed. Quantification of β-catenin and CCL28 was performed using ImageJ software for gray scale analysis statistics.

10. B16 Melanoma and 4T1 Breast Cancer Subcutaneous Xenograft Tumor Model

B16 or 4T1 cells in logarithmic growth phase were taken, and the cell concentration was adjusted using PBS to 3×10⁶ cells/ml. 6-week old WT female mice were taken. Lateral mouse hair of the right hind limb of the mice were cut off using Ophthalmic scissor and disinfected with 75% alcohol. Each mouse was injected with 0.1 ml cell suspension subcutaneously. When a tumor with a diameter of 5 mm is subsequently observed, it is recorded as the starting point of time. The tumor size was measured and calculated with vernier caliper.

11. Statistical Analysis

The data are expressed as mean±SD. Statistical significance between groups was calculated by the two-tailed unpaired Student t-test (GraphPad Prism). P<0.05 is considered to be statistically significant.

### Example 1. β-Catenin Signaling Pathway Induces CCL28 Expression in Gastric Cancer

In this example, the effects of Wnt/β-catenin signaling pathway on chemokine expression in gastric cancer cell lines were detected. The method is as follows: 1) overexpressing β-catenin in gastric cancer cell SGC7901, and expressing mutant β-catenin (i.e. S33Y.β-catenin) on GSK3β phosphorylation site mutation using plasmid vector, thereby realizing continuous activation of β-catenin, and then detecting changes in chemokine expression using qPCR; 2) In human gastric cancer cell lines AGS and SGC7901 respectively, through knocking down or overexpressing β-catenin, WB was used to detect the changes of CCL28 expression level at the protein level. 3) in clinical gastric cancer tissue samples, after immunohistochemical staining, verifying the correlation between β-catenin and CCL28 by statistical analysis of the positive rates of β-catenin and CCL28.

Results:

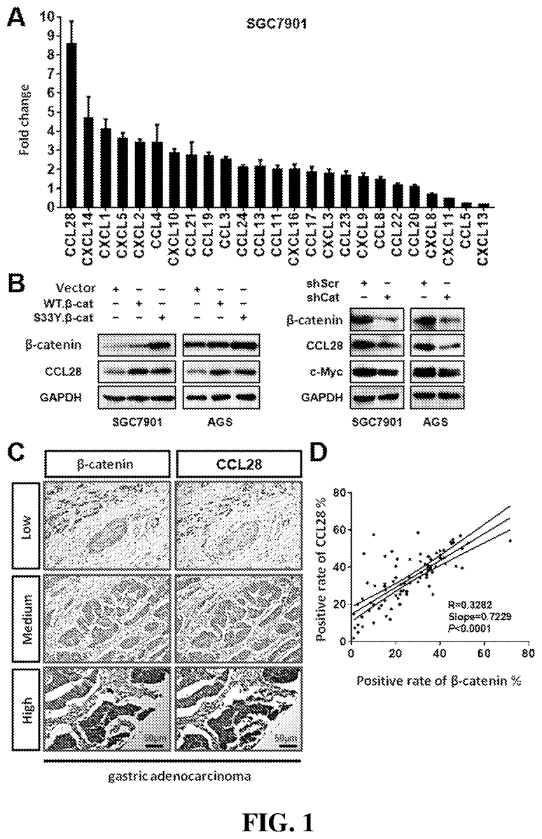

qPCR results show that CCL28 is the chemokine with the most significant change after overexpression of β-catenin, compared to the control plasmid (FIG. 1A).

In SGC7901 and AGS human gastric cancer cell lines (derived from the cell bank of Chinese Academy of Sciences), overexpression of wild-type (WT) or degradation-resistant mutant S33Y.β-catenin also increases CCL28 protein levels (FIG. 1B). In contrast, knockdown of β-catenin in both cell lines results in decreased CCL28 protein levels (FIG. 1B).

### Example 2. CCL28 is a Direct Target Gene of β-Catenin Signaling Pathway in Human Gastric Cancer Cells

In this Example, the correlation between CCL28 and β-catenin signaling pathway structure is analyzed in combination with bioinformatics, and then the relationship between the two is verified in the cell model. The methods are as follows: finding three potential binding sites of β-catenin/TCF transcription complexes in the promoter region of CCL28 by using Bioinformatics, analyzing whether β-catenin is enriched at the potential binding sites by using chromatin immunoprecipitation technology, cloning CCL28 promoter and CCL28 promoter sequence mutated with potential binding site sequence into luciferase reporter vector to check the transcription activity of CCL28 promoter. After using β-catenin/TCF, the transcriptional activity of CCL28 promoter was detected after shRNA knockdown of TCF/LEF transcription factor family members or overexpression of TCF/LEF members.

Results:

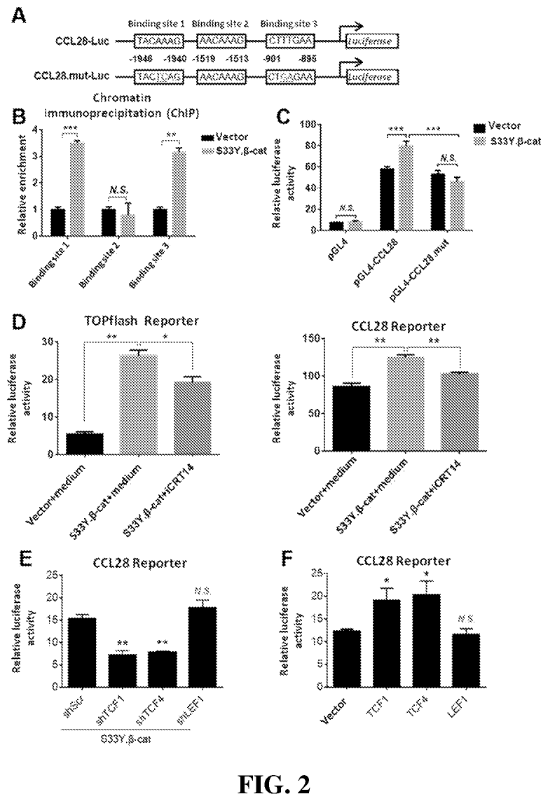

Bioinformatics analysis reveals three potential binding regions of the β-catenin/TCF transcription complex in the promoter region of the CCL28 gene (FIG. 2A).

In human gastric cancer cell SGC7901, β-catenin is found to be bound to promoter sites 1 and 3 using chromatin immunoprecipitation analysis (FIG. 2B).

After mutating the two binding site sequences, the up-regulation effect of β-catenin on CCL28 promoter activity disappears (FIG. 2C), which indicates that β-catenin directly regulates the transcription of CCL28 gene by binding sites on the promoter of CCL28.

The use of the Wnt signaling pathway inhibitor iCRT14 also reduces the up-regulation effect of β-catenin on CCL28 promoter activity (FIG. 2D). Treatment with Wnt signaling pathway inhibitor iCRT14 hinders the binding of β-catenin and TCF in cells and the binding of β-catenin/TCF transcription complex on promoter DNA, thus inhibiting Wnt signaling pathway. The transcriptional activity of CCL28, a targeting gene of Wnt signaling pathway, is also inhibited. By detecting CCL28 promoter activity after shRNA knockdown or gene overexpression, it is found that transcription factors TCF1 and TCF4 cooperate with β-catenin to regulate the transcription of CCL28 gene, but not LEF1.

### Example 3. Wnt Signaling Pathway Inhibitor iCRT14 Inhibits Expression of CCL28 in Mouse In Situ Gastric Cancer

In this example, in situ gastric cancer in mice was induced using H. felis infection and carcinogen MNU, and then the expression level of CCL28 protein in the stomach was detected by intraperitoneal injection of the Wnt signaling pathway inhibitor iCRT14.

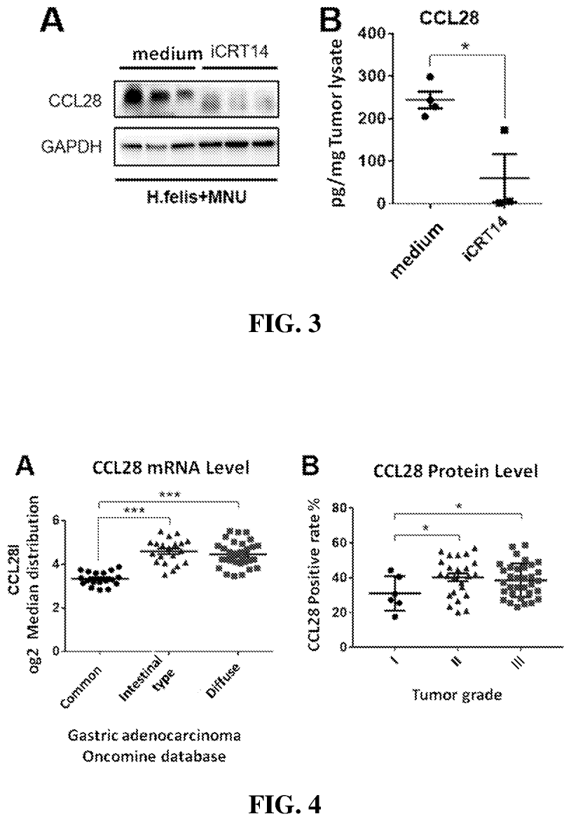

The results are shown in FIG. 3. Both Western blotting (FIG. 3A) and ELISA (FIG. 3B) analysis show that iCRT14 significantly reduces the expression of CCL28 protein in the stomach, indicating that CCL28 expression is also regulated by β-catenin pathway in vivo.

### Example 4. CCL28 is Related to the Development of Gastric Cancer

In this example, immunohistochemical analysis further analyzes the correlation between expression levels of CCL28 and β-catenin. The results show that there is a significant positive correlation between expression level of β-catenin and that of CCL28 in different degrees of gastric cancer samples (FIGS. 1C and 1D).

Based on the Oncomine database, CCL28 and the development degree of gastric cancer were further analyzed. The results show that the mRNA levels of CCL28 in intestinal type and diffuse type gastric cancer are higher than that in normal tissues (FIG. 4A). Immunohistochemical staining of tissue samples from gastric cancer patients also show that the protein level of CCL28 is also higher in tumors with higher malignant grade (FIG. 4B).

In brief, these results suggest that CCL28 is a target gene of the Wnt/β-catenin signaling pathway in gastric cancer cells, suggesting that CCL28 is a potential pathogenic factor in gastric cancer. In addition, the positive rate of CCL28 becomes higher as gastric cancer develops or malignant grade increases.

### Example 5. Gastric Cancer Cells Activated by the β-Catenin Signaling Pathway Induce the Expression of CCL28 to Achieve the Migration of Regulatory T Cells

In this example, tumor cells with activated Wnt/β-catenin were detected by in vitro migration experiments whether it can recruit human Treg cells through CCL28. The method is as follows: constructing a gastric cancer cell line that overexpresses β-catenin in a gastric cancer cell line SGC7901, or overexpresses β-catenin while CCL28 being knocked down using shRNA, collecting the supernatant of the cell culture medium, spreading it in the lower layer of transwell, and spreading peripheral blood mononuclear cells at 1×10⁶ in the upper chamber, culturing in an incubator at 37° C. for 4 hours, cells migrated to the lower layer were collected, the proportion and number of immune cells were analyzed by flow cytometry.

Results:

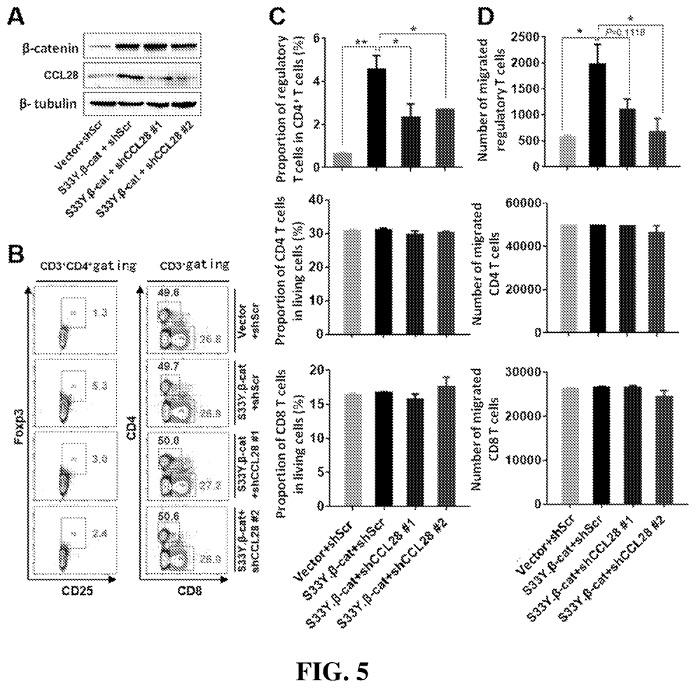

Knockdown of S33Y.β-catenin-induced CCL28 by shRNA in SGC7901 cells (FIG. 5A).

In the migration experiment, peripheral blood mononuclear cells that migrated to the lower layer were analyzed by flow method for changes in immune cells (FIG. 5B).

Under the condition that gastric cancer cell line SGC7901 overexpresses β-catenin, the proportion and absolute number of regulatory T cells in CD4⁺ T cells increased significantly, while it decreased when β-catenin being knocked down (FIG. 5C, D). These results suggest that β-catenin-activated gastric tumor cells recruit Treg cells through CCL28 in vitro.

The flow results did not show any difference in total CD4⁺ or CD8⁺ T cell recruitment (FIG. 3C, D), suggesting the unique effect of β-catenin/CCL28 on Treg.

### Example 6. CCL28 Antibody Treatment Inhibits the Progression of H. felis/MNU-Induced Gastric Cancer

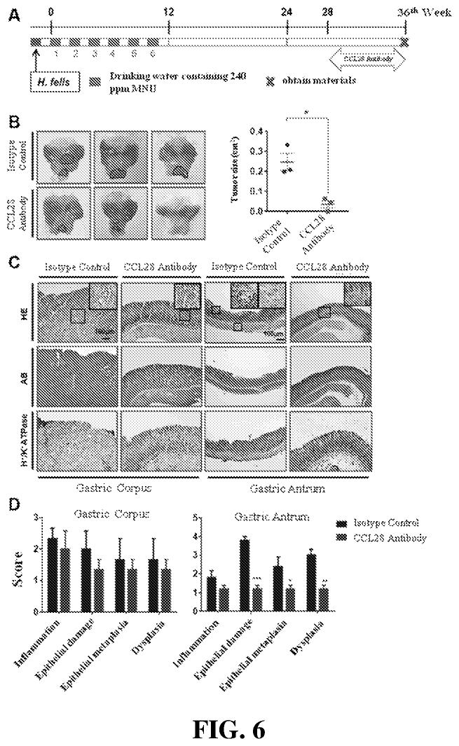

In this example, H. felis/MNU gastric cancer model animals were used, and the model mice were administered with CCL28 monoclonal antibody once a week to block the activity of CCL28 at the 29th week after the start of MNU treatment (indicated in the figure is the end of the 28th week and the beginning of the 29th week). The experimental scheme is shown in FIG. 6A.

Results: After 8 weeks of antibody treatment, the tumor size in the stomach of the CCL28 antibody-treated mouse model was significantly reduced (FIG. 6B). Histological analysis showed significant changes in dysplasia and intestinal transformation with CCL28 antibody therapy (FIG. 6C). It can be seen from immunohistochemical analysis of H+K+ATPase that after treatment with CCL28 antibody, the phenomenon of parietal cell loss was significantly relieved in the gastric antrum (FIG. 6D). Which suggests that inhibition of CCL28 can effectively alleviate the progression of gastric cancer.

### Example 7. CCL28 Antibody Treatment Alleviates the Inhibition of the Immune Microenvironment

In this example, for the model animals treated with CCL28 antibody in Example 6, FACS analysis was performed on mouse gastric tumor samples at the end of the 36th week.

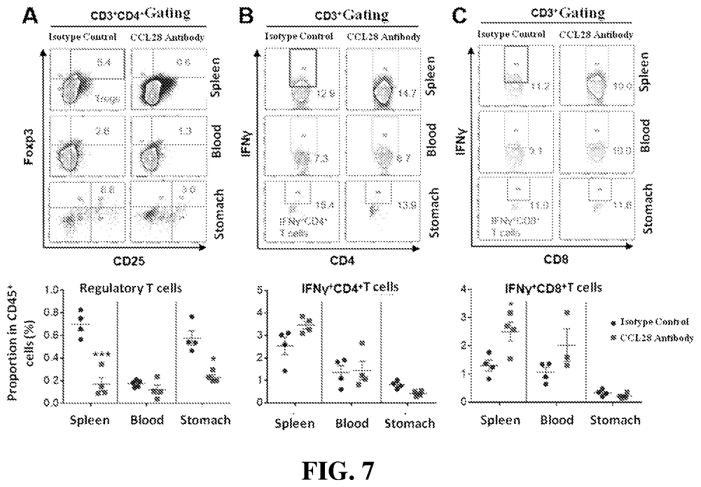

The results were shown in FIG. 7. FACS analysis confirmed a significant reduction of regulatory T (Treg) cells in the stomach of CCL28 antibody-treated mice (FIG. 7A). Interestingly, anti-CCL28 treatment also reduces the rate of Treg cells in the spleen, but does not reduce the rate of Treg cells in the blood, suggesting the presence of a chemokine CCL28-dependent Treg recruitment mechanism in the spleen and stomach. In addition to the direct effect on the stomach, the role of CCL28 antibody treatment in the spleen can also improve the overall immune response, because effective immunotherapy requires peripheral immune activation and coordination. In conclusion, these data suggest that anti-CCL28 therapy effectively inhibits the progression of H. felis/MNU-induced gastric cancer by inhibiting the recruitment of Treg.

What is worth mentioning is that by detecting CD4⁺/CD8⁺ T cells of IFN γ+ (FIG. 7B, C), the results show that these effector T cells are improved to a certain extent in spleen or peripheral blood, but no obvious changes in stomach, indicating that CCL28 antibody treatment can improve the activity of peripheral immunity, however, there is little effect on the process of infiltration of effector T cells into solid tumors.

### Example 8. CCL28 Antibody has No Significant Therapeutic Effect on Melanoma and Breast Cancer

In this example, the therapeutic effects of CCL28 antibody treatment on other cancers were verified. The method was as follows: Mouse melanoma cells B16 and breast cancer cells 4T1 were used to establish subcutaneous xenograft tumors: the cells in the logarithmic growth phase were taken and resuspended with PBS after trypsin digestion. The 6-8-week C57BL/6 mice were anesthetized, the underarms of the forelimbs of the mice were treated with shaving apparatus, and the exposed skins were disinfected with 75% alcohol. The cell suspension with adjusted cell density was inoculated into the armpit of the forelimbs of mice, and the tumor growth was observed and recorded every day. Wherein B16 cells 5×10⁵ cells/mouse, 4T1 cells 1×10⁶ cells/mouse. B16 xenograft tumor mice and 4T1 xenograft tumor mice were treated with CCL28 antibody on day 3-7 after cell inoculation, and the treatment method and dosage were the same as in Example 6.

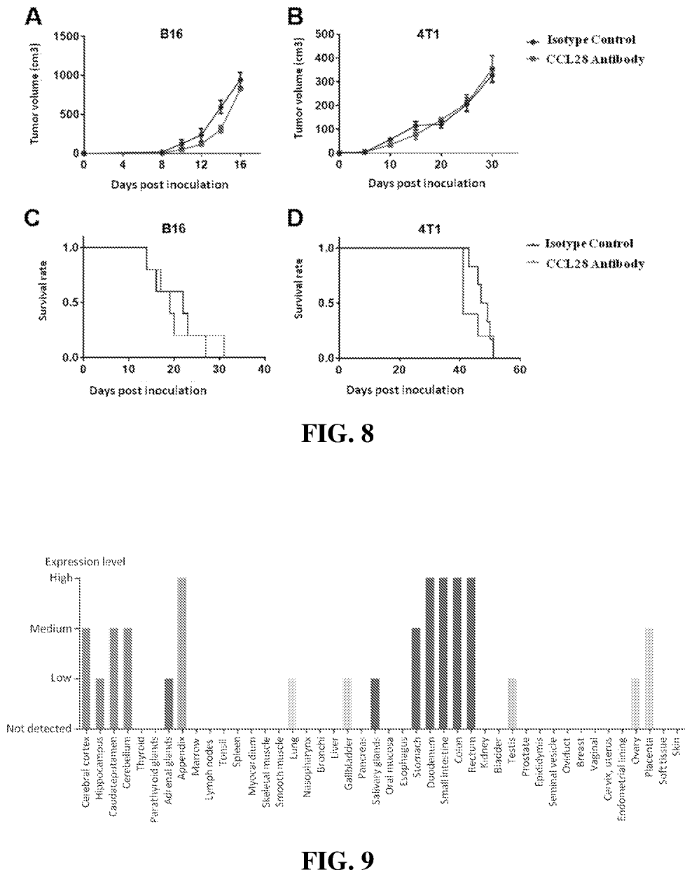

The results showed that CCL28 antibody did not show significant therapeutic efficacy in the growth (FIGS. 8A and B) and survival (FIGS. 8C and D) of mouse xenograft tumors after treatment with CCL28 antibody or control IgG antibody.

### SUMMARY AND DISCUSSION

The present inventors discovered a synergistic effect of Wnt/β-catenin (one of the common oncogene pathways in cancer) with the expression of CCL28 in gastric cancer cells, and this effect then increases the number of regulatory T cells in the tumor. Through a large number of subsequent inventive studies, the present inventors unexpectedly found that in gastric cancer, the use of monoclonal blocking antibody of CCL28 can effectively reduce the infiltration of regulatory T cells in the tumor and inhibit the development of the tumor. These inventive research results suggest that Wnt/β-catenin not only directly regulates immune regulation mechanism except cell proliferation and survival, but also provides a new immunotherapy target CCL28.

More surprisingly, the present invention demonstrated through comparative experiments that the linkage mechanism between Wnt/β-catenin and CCL28 is uniquely present in a special pathological scenario—gastric cancer, but not present in other tumors, even other solid tumors such as melanoma cell B16 and breast cancer cell 4T1 models.

On the other hand, recent studies suggest that regulating T cell apoptosis may lead to a higher immunosuppression, and immunotherapeutic approaches that directly target regulatory T cells for apoptosis may not achieve optimal therapeutic effects. However, the present invention establishes a method for blocking the CCL28 chemotactic pathway, thereby targeting the migration process of regulatory T cells to the tumor and not affecting the apoptosis of the T cells, which may provide a more novel and effective means for the immunotherapy targeting regulatory T cells. Furthermore, for patients suffering from gastric cancer and other immunotherapy for a long time, low autoimmunity and derived diseases thereof are important causes of death. Therefore, the method provided by the present invention is not only able to effectively inhibit gastric cancer metastasis, while not reduce the patient's own immunity, but also improves the overall immunity.

In addition, the present invention also confirms the correlation of CCL28 and its receptor level with the occurrence and severity of gastric cancer, through a large number of clinical data for the first time, thus developing a detection system for an accurate insight into the occurrence, metastasis and prognosis of gastric cancer.

In general, CCL28 blocking therapy has great clinical potential in cancer treatment and may improve the efficacy of other immunotherapy.

All documents mentioned in the present invention are cited as references in this application, just as each document is individually cited as a reference. In addition, it should be understood that, after reading the above teaching content of the present invention, those skilled in the art can make various changes or modifications to the present invention, and these equivalent forms also fall within the scope defined by the appended claims of the present application.

1. A method for detecting gastric cancer, wherein the gastric cancer is a type of gastric cancer wherein both Wnt/β-catenin signal pathway and chemokine CCL28 related molecule expression are abnormal up-regulated at the same time; and said method comprises a step of:
using a CCL28 gene or CCL28 protein, or a detection reagent thereof, or using a kit prepared from a CCL28 gene or CCL28 protein, or a detection reagent thereof to detect the gastric cancer.

2. The method of claim 1, wherein the pathological manifestations of the gastric cancer have one or more pathological manifestations selected from the group consisting of:
(1) up-regulated Wnt/β-catenin signaling pathway-related expression in gastric tissues;
(2) up-regulated chemokine CCL28 related expression in gastric tissue;
(3) gastric tumor;
(4) loss of gastric parietal cells; and
(5) increase of regulatory T cells of gastric tissues.

3. The method of claim 1, wherein the gastric cancer comprises an in situ gastric cancer, an intestinal gastric cancer, and a diffuse gastric cancer.

4. The method of claim 2, wherein the Wnt/β-catenin signaling pathway related expression includes the expression level of the transcription factor TCF1 and TCF4, the content of β-catenin, the level of β-catenin nuclear ectopia, the transcriptional activity of β-catenin/TCF4 complex, the level of GSK3β phosphorylation, or combinations thereof.

5. The method of claim 1, wherein the chemokine CCL28 related molecule comprises a gene, protein of CCL28 and a receptor thereof or a combination thereof.

6. The method of claim 1, wherein the reagent comprises specific primer, specific antibody, probe and/or chip of the CCL28-related molecule.

7. A kit for detecting gastric cancer, the kit comprises a container containing a detection reagent for detecting CCL28-related protein or mRNA; and a label or instruction indicating that the kit is used for detecting gastric cancer.

8. The kit of claim 7, wherein the gastric cancer is a type of gastric cancer wherein both Wnt/β-catenin signal pathway and chemokine CCL28 related molecule expression are abnormal up-regulated at the same time.

9. The kit of claim 7, wherein the detection reagent comprises specific primer, specific antibody, probe and/or chip.

10. The kit of claim 7, wherein the contents indicated in the label or instruction are selected from the group consisting of:
a) when the ratio of the mRNA expression A1 of the CCL28-related protein of a testing subject to the mRNA expression A0 of the CCL28-related protein of the para-carcinoma tissue (A1/A0) is ≥2, prompting that the probability of gastric cancer of the testing subject is higher than that of the general population;
b) when the ratio of the mRNA expression of the CCL28-related protein of a testing subject to the mRNA expression of the CCL28-related protein of the para-carcinoma tissue A1/A0 is ≥2, the higher A1/A0 ratio indicating the higher malignant degrees of the gastric cancer of the testing subject;
c) when the ratio of the mRNA expression of the CCL28-related protein of a testing subject to the mRNA expression of the CCL28-related protein of the para-carcinoma tissue A1/A0 is ≥2, the higher A1/A0 ratio indicating poorer prognosis and higher metastasis rate of the gastric cancer of the testing subject;

11-13. (canceled)

14. A method for screening candidate compounds for treating cancers, including the steps of:
(a) in a testing group, adding a testing compound to the cell culture system, and observing the expression and/or activity of CCL28-related molecules in the cells of the testing group; in a control group, not adding the testing compound to the culture system of the same cell, and observing the expression and/or activity of CCL28-related molecules in the cells of the control group;
wherein if the expression and/or activity of CCL28-related molecules of the cells of the testing group is lower than that of the control group, indicating that the testing compound is a candidate compound for treating cancer having inhibitory effect on the expression and/or activity of CCL28-related molecules.

15. A method for inhibiting or treating gastric cancer, comprising the steps of: administering a safe and effective amount of a CCL28 inhibitor or a CCL28 related molecule inhibitor to a subject in need; the gastric cancer is a type of gastric cancer wherein both Wnt/β-catenin signal pathway and chemokine CCL28 related molecule expression are abnormal up-regulated at the same time.

16. The method of claim 15, wherein the CCL28 inhibitor is selected from the group consisting of an antibody or small molecule inhibitor targeting CCL28 and/or the receptor protein thereof; a targeting nucleic acid molecule or gene editor targeting CCL28 and/or receptor gene thereof; or combinations thereof.
